(19) United States
(12) Patent Application Publication
(10) Pub. No.: US 2013/0011843 A1
(43) Pub. Date: Jan. 10, 2013

(54) TISSUE SEPARATION METHOD

(75) Inventor: Ulrich Hannappel, Slater, IA (US)

(73) Assignee: SYNGENTA PARTICIPATIONS AG, Basel (CH)

(21) Appl. No.: 13/636,694

(22) PCT Filed: Mar. 23, 2011
(86) PCT No.: PCT/US11/29668

§ 371 Date: Sep. 23, 2012

Related U.S. Application Data
(60) Provisional application No. 61/341,101, filed on Mar. 26, 2010.

(51) Int. Cl. A01C 1/06; B03B 1/00; C12Q 1/68

(52) U.S. Cl. 435/6.11; 209/9

(57) ABSTRACT

The invention relates to a method for non-destructively sampling individual seeds in a population of seeds. In one embodiment, the invention relates to an efficient, high throughput method for removing contaminating tissue from the other seed material. The methods of the invention are useful for determining the genotype of a seed and the detection of a genetic marker or genetic trait. The methods of the invention comprise removing maternal tissue, such as seed coat or pericarp from the seed, and analyzing the remainder of the seed. The methods of the invention reduce the degree of ambiguity in the genetic tests because complicating maternal tissue has been removed.

### CROSS REFERENCE TO RELATED APPLICATIONS

This application claims benefit of U.S. Provisional Application Ser. No. 61/341,101 filed Mar. 26, 2010 and Patent Cooperation Treaty International Patent Application Ser. No. PCT/US11/29668 filed Mar. 23, 2011, which are incorporated herein by reference in their entirety.

### FIELD

The invention is a method for non-destructively sampling individual seeds in a population of seeds. In one embodiment, the invention is an efficient, high throughput method for the removal of undesired seed tissue from desired seed tissue. In another embodiment, the method comprises removing maternal tissue, from a seed, and analzying some of the remaining portion of the seed for a genetic marker or genetic trait of interest or other seed phenotype or genotype such as oil, starch, genetically modified trait, a DNA or RNA sequence or protein.

### BACKGROUND

Amplification of endosperm DNA from seeds has been known for at least 15 years as shown by Chunwongse et al., Theoretical Applied Genetics 86:694-698 (1993). High throughput genotyping systems which genotype seed DNA, are used in testing many varieties of seed including maize, wheat, vegetables, flower, sunflower, sugar beet, rice, soy and others. High throughput seed chipping devices are efficient because undesired seeds can be identified and discarded, and desired seeds can be identified and retained. Seed chips are suitable seed DNA samples for genotyping and can be the subject of marker assisted plant selections for most of the breeding processes used in plant breeding programs. Genotyping of seed DNA, from a portion of the seed that is chipped off, while leaving a viable seed, before planting that seed is beneficial in many ways. The testing of the chipped seed portion allows the identification and selection of the viable seed with preferred genotypes and it also allows the identification and elimination of the less preferred seed genotypes.

However, determining the genotype of a seed embryo, from the seed chip, can be complicated due to the presence of maternal tissues like the seed coat. These tissues add ambiguity to the test results and result in the selection of inappropriate seeds. This ambiguity can be eliminated by removal of the maternal tissue prior to seed DNA testing.

Removal of maternal tissue from a seed sample is often not easy because it requires the removal of the outer seed coating, the pericarp of a seed. The relatively small size of most seeds makes separation and removal of maternal seed coats difficult, or at best, time consuming and laborious. Corn pericarp, which is an example of maternal seed tissue, is the mature ovarian female tissue of the seed. The seed coat or pericarp's function is to protect the interior endosperm and embryo from diseases and moisture loss. To accomplish this function, the pericarp is usually a layer, several cells thick and tightly adhered to the interior part of the seed. The hard protective nature of the maternal seed coat is tough to remove, thereby, making it challenging to analyze the remainder of the seed material.

For example, corn and soy processing applications frequently remove the seed's maternal tissue because the make up of pericarp detrimentally affects the nature and composition of the end product. Therefore, a number of chemical solvents and soaking processes have been developed by the processing industry to remove the tough maternal coat tissues. However, these industrial processes are time consuming and not designed to protect the integrity of the seed endosperm DNA tissue. Thus these processes are less than ideal for preserving seed DNA in the non-maternal tissues of a seed.

Thus, there remains a need for an automated, high throughput, efficient method for non-destructively removing maternal seed tissue, such as a seed coat, from individual seeds or seed pieces, while preserving the seed in a testable form.

### BRIEF SUMMARY

The methods of the invention increase the efficiency of selecting seeds that have a desired trait or genotype, or phenotype in a population of seeds. The invention also relates to an efficient, high throughput method for testing seed material. The invention is particularly useful for testing for purity standards for genetically modified traits. But this invention is also useful for testing adventitious genetically modified organisms presence, for marker-assisted selection in breeding, chromosomal patterns and number and genetic purity of germplasm. In another embodiment, the invention relates to a method for non-destructively sampling individual seeds in a population of seeds and then selecting seeds from this population based on the results of the tests. The seeds can be analyzed for a specific allele, haplotype, genetic locus, or a genetic trait, phenotype or genotype or other seed components of interest that are subject to detection in the seeds.

In one embodiment, the invention relates to a method of selecting seed having a trait, the method comprising: (a) coating seed with an coating, (b) separating, in a non-destructive manner, at least a portion of coated seed coat from an individual seed, (b) separating the coated seed coat or portion thereof from the seed or portion thereof; (c) analyzing the coat-free seed or seed portion thereof for the presence or absence of at least one trait of interest; and (d) selecting seeds based on the analysis of said coat free seeds or seed portions. The methods can be performed without affecting the germination viability of the seeds. In one embodiment, the coated seed comprises a seed coated in metallic paint, metal or a metallic covering. The method can analyze the genetic components of the coat-free seed or seed portion thereof for the presence or absence of at least one trait of interest. The method can analyze sequences of genetic material

In one embodiment, the invention relates to a method of selecting seeds in a population having a desired trait, the method comprising: (a) applying a coating to at least a portion of a seed; (b) removing, in a non-destructive manner, a seed chip comprising at least a portion of seed coat from the coated seed, (c) separating the seed coat from the seed chip; (d) analyzing the coat-free seed chip for the presence or absence of at least one trait of interest; and (e) selecting seeds for a seed population based on the presence or absence of at least one trait of interest. The methods can be performed without affecting the germination viability of the seeds. The coating is selected so that germination viability of the seed is not affected.

In one embodiment, the coating is responsive to magnetic forces. The coating can be any substance that allows separation of the unwanted material from the desired material including but not limited to metallic paint, metal, metallic coating.

In one embodiment, the seed coat is separated from the seed chip using an attractant including but not limited to a magnet, a magnetic plate or a magnetic liquid.

In one embodiment, the invention relates to a method of analyzing seeds for a trait, the method comprising: (a) applying a coating to at least a portion of a seed; (b) loosening the coated seed coat from at least a portion of the seed; (c) separating the coated seed coat from the portion of the seed (d) analyzing the coat-free seed or seed portion for the presence or absence of at least one trait. This method also can have a step of selecting or deselecting seed based on the presence or absence of at least one trait of interest. In at least one embodiment of this invention separating the coated seed coat employs an attractant force. The method may comprise a coating wherein said coating is a magnetic coating. The step of separating the coated seed coat employs an attractant force in automated high throughput system of separating seed coat from seed or a portion thereof.

In one embodiment, the invention relates to a method of selecting seeds in a population wherein at least some seeds have at least one trait of interest, the method comprising: (a) applying a coating to at least a portion of a seed; (b) removing, in a non-destructive manner, a seed chip comprising at least a portion of coated seed coat from the coated seed; (c) separating the seed coat from the seed chip; (d) analyzing the coat-free seed chip for the presence or absence of at least one trait; and (f) selecting seeds from said seed population based on the presence or absence of at least one trait of interest.

In another embodiment, the methods comprise planting selected seeds. The method of the invention comprises cultivating plants from the selected seeds. In yet another embodiment, seeds can be harvested from the cultivated plants. Products of the method of the present invention comprise a seed or a portion of a seed without a seed coat. Another product of the method is a coated viable seed comprising a seed coat portion and a non seed coat portion, wherein the non seed coat portion also lacks the coating. Another product of the method is a seed coat having an inner surface and an outer surface, wherein the outer surface is coated with a magnetic material. Another product of the method is a portion of a seed coat having an inner surface, wherein said seed coat inner surface is adapted to engage with at least a portion of seed which is not seed coat, and wherein said portion of said seed coat inner surface is detached from said portion of seed and said outer seed coat surface is coated.

In another embodiment, the invention relates to a method for removing at least a portion of seed coat from at least a portion of seed in an automated process. The invention relates to a method for removing at least a portion of seed coat from at least a portion of a seed comprising: (a) applying a coating to at least a portion of a seed; (b) loosening seed coat from at least the portion of the seed that is coated; (c) removing at least a portion of the coated seed coat; and, (d) retaining the remainder of the seed after removing the coated seed coat.

An advantage of the invention is a rapid, efficient, optionally automated, high throughput method for selecting seeds with a trait of interest.

An advantage of the invention is a clean analysis of the genotype of a seed, without seed coat confounding the analysis. This analysis of the genotype is nondestructive to seed viability, and the analysis occurs prior to planting seed, thereby only the selected seed are planted, which results in reducing the time, cost and labor associated with producing seeds with a desired trait.

An advantage of the invention is the use of a coating, which does not damage DNA, to remove contaminate tissue, such as maternal seed coat or pericarp, and increase the accuracy of the genetic analysis of the seed.

An advantage of the invention is it can produce three seed components for testing, genotyping, or testing and genotyping a seed, a seed chip, and a seed coat. One method testing or genotyping process can be used to give efficient and accurate analysis of all three types of seed component samples.

An advantage of the invention is the preservation of the seed coat which can be employed for testing, genotyping or testing and genotyping of its DNA to determine the maternal parent's genotype.

An advantage of the invention is it allows for separated seed component samples. This allows the seed or seed chip to be tested by extracting starch, meal, germ, flour, ethanol, oil, protein from the seed material after removal of the detached seed coat.

### BRIEF DESCRIPTION OF THE DRAWINGS

FIG. 1 is an allelic discrimination plot that has homozygous and heterozygous alleles that are not clearly distinguishable, by the ordinary skilled person; and an automated analysis of this allelic discrimination plot would not be possible.

FIG. 2 is an allelic discrimination plot using the same sample as the sample used in the allelic discrimination plot shown in FIG. 1 except the seed was tested without the seed coat which has been excluded from the sample tested.

FIG. 3 three sets of soybean seeds are shown one is an uncoated control (left), the next set of seeds are lightly coated with magnetic paint (middle), the last set of seeds are heavily coated with magnetic paint (right).

FIG. 4 is a 96 well block is shown with each well containing a detached seed coat and cotyledon tissue (present as a seed chip), the seed coat and cotyledon tissue have been separated by exposure of the seed material to high temperature.

FIG. 5 shows a 96 prong magnet, each magnet has a enlarged head attached to a shaft. Each magnet is adapted to fit within an individual well.

FIG. 6 shows a 96 prong magnet inserted into the 96 well block containing seed coat and cotyledon tissue (seed coat and tissue not shown).

FIG. 7 shows the coated seed coat which has been extracted from the well block containing cotyledon tissue, the coated seed coat is held by attractant force to the magnetic prongs.

FIG. 8 shows a close up photograph of the coated seed coat magnetically engaged with the magnetic prongs.

FIG. 9 shows the 96 well block of FIG. 6 after the magnetic prongs shown in FIG. 7 and FIG. 8 have been introduced into the well containing seed material and coated detached seed coats. The prongs show attracted coated seed coat adhered on to the prong. This prong has been removed from the well with the attracted coated seed coat while leaving the uncoated detached cotyledon tissues remaining in the 96 wells.

FIG. 10 shows another design of another type of prong magnet with a different number of prongs and different shape of prong, but still adapted for selectively attracting coated seed material from a mixture of coated and uncoated seed material.

FIG. 11 shows another method of employing the prong magnet. The prong magnet is placed below the wells of a 96 well block. Each prong attracts magnetically coated seed particles from more than one well to a magnetic prong at the bottom of the well block; the well block can be inverted to remove the uncoated seed material through the top opening, while the coated seed material is held to an enclosed bottom of the well by the strength of the magnetic attraction of the coating on the seed to the magnet.

FIG. 12 shows a block or flat magnet which is of sufficient strength to attract the coated seed material.

FIG. 13 shows a flat magnet placed over a 96 well block to immobilize the attracted coated seed material proximate the magnet.

### DETAILED DESCRIPTION

### Definitions:

The numerical ranges in this disclosure are approximate, and thus may include values outside of the range unless otherwise indicated. Numerical ranges include all values from and including the lower and the upper values, in increments of one unit, provided that there is a separation of at least two units between any lower value and any higher value. As an example, if a compositional, physical or other property, such as, for example, molecular weight, viscosity, melt index, etc., is from 100 to 1,000, it is intended that all individual values, such as 100, 101, 102, etc., and sub ranges, such as 100 to 144, 155 to 170, 197 to 200, etc., are expressly enumerated. For ranges containing values which are less than one or containing fractional numbers greater than one (e.g., 1.1, 1.5, etc.), one unit is considered to be 0.0001, 0.001, 0.01 or 0.1, as appropriate. For ranges containing single digit numbers less than ten (e.g., 1 to 5), one unit is typically considered to be 0.1. These are only examples of what is specifically intended, and all possible combinations of numerical values between the lowest value and the highest value enumerated, are to be considered to be expressly stated in this disclosure. Numerical ranges are provided within this disclosure for, among other things, relative amounts of components in a mixture, and various temperature and other parameter ranges recited in the methods.

The term “attractant” refers to any substance or component that attracts a desired substance or component with sufficient force to permit separation of the desired substance or component.

The term “contaminate tissue” refers to undesired tissue for a particular analysis.

The term “coating” or “coated” refers to any substance or component added to a seed, either alone or in a mixture of components, wherein the substance or component has a characteristic that attracts or binds to an attractant.

The term “plant” includes monocotyledenous plants, dicotyledenous plants and transgenic plants.

The invention relates to an efficient, high throughput method for testing seed material. The invention is particularly useful for work requiring high purity standards like, for example, work with genetically modified traits. The required purity of seed in seed packaging is approximately 95% purity of the seed. This requires that 95% or more of the plants are the variety and are not off types. Limiting seed off types is critically important to the end users. Purity is particularly important when seeds carry an crucial trait, such as a tolerance or resistance to a pest, herbicide or pathogen. Thus if the seed is carrying an insect resistant GM trait then 95% or more of the seed must contain an effective amount of this trait. If the trait is herbicide tolerance then the negative consequences of impurities are highly visible to the end grower, because use of a herbicide spray results in the death of the impurities. Therefore, seed purity levels are stringently tested. Assays are used to test seedlings, grown from seed for the desired profile whether it is a genotype, phenotype or genetically engineered trait. The detected number of off type seedlings (seedlings without the desired profile) are used to extrapolate the total percentage of seed that may be off types in the seed lot. Although, this system can be very accurate when the impurity is randomly distributed throughout the seed lot, if the impurity is not randomly spread throughout the seed lot then the accuracy of the determined purity is questionable.

Generally, this invention provides an improved seed chip test with rapid results for a larger sampling or all of the seeds in the seed lot if total testing is desired. The improvement of the present invention assists in negating some of the potential inaccuracies of the previous testing methods. Use of a seed chip, in contrast, to use of a seedling may result in less of the detectible chemical or compound for assaying. And seeds, unlike seedlings contain a more diverse mix of DNA. The seed of maize for example has different ploidys in different part of the seed such as the embryo, endosperm and seed coat. The seed coat is totally maternal, the embryo is both maternal and paternal and the endosperm has two doses maternal and one dose paternal making its ploidy triploid. If the characteristic which the assay is detecting is on the male parent, the embryo and endosperm have identical alleles, and expression in the endosperm will not bias the result. If however, the characteristic is on the female parent, an inaccurate, or unclear assay result may be obtained if the allele is expressed in the seed coat.

If the seed is from a selfing species, then negative traits can be unnoticed because heterozygote's self and when this segregation occurs for the trait then a higher trait purity is detected then is actual.

Trait testing is often complex, because multiple traits such as triple/quad stacks of traits can be common. It would be useful to be able to test all traits from the same seed chip, without the confounding, complexity added by the extra seed coat information. If it was simple to remove a seed coat prior to testing it would be removed. However, seed coats are difficult to detach and then separate from the seed material that is useful for testing. The present invention provides an automated high through put method for separating seed coats from other seed material. The present invention provides a coatless seed or seed portion which is useful for testing methods associated with identifying events, marker-assisted selection and breeding and genetic purity of seed material.

The invention also provides an automated, efficient method for non-destructively sampling of individual seeds, without seed coats, in a population of seeds. Nondestructive seed sampling methods can correlate the seed and the seed sample, from which the seed coat is removed, in a high-throughput platform. This platform tests seed sample, which has been separated from its seed coat, for certain characteristic(s) and the correlated seeds populations of seeds having the characteristic(s), such as and event, marker or genotype are selected for further use. With the accuracy of the coatless seed chip test results, seeds are selected and bulked quickly.

In an embodiment, the method comprises removing contaminate seed tissue from desired tissue in a seed. In one embodiment, the method comprises removing maternal seed tissue contaminate from endosperm-derived tissue in seed samples. In yet another embodiment, the invention provides a method for automated separation of maternal seed coat DNA from the seed DNA, analyzing and genotyping the seed DNA. And optionally. comprises a method for analyzing and genotyping the maternal seed coat DNA.

In one embodiment, the invention relates to an automated, high throughput method for removing contaminate or unwanted material from desired seed tissue. In this embodiment, the method comprises the step of extracting pericarp/seed coat DNA from seed tissue. Analysis of DNA extracted from the resulting tissue provides a clear genotype of the future plant. The improved genotyping of seed genetic characteristics allows for improved, more efficient marker-assisted breeding programs.

The seed can be derived from a monocotyledenous plant or a dicotyledenous plant. Nonlimiting examples of a monocotyledonous plant is, turf, turf grass, cereals, maize, rice, oat, wheat, barley, sorghum, orchid, iris, lily, onion, banana, sugarcane, sorghum, and palm. Nonlimiting examples of a dicotyledenous plant is avocado, potato, tobacco, tomato, sugarbeet, broccoli, cassava, sweet potato, pepper, canola, rape seed, cotton, melons, cucumbers, poinsettia, legumes, alfalfa, soybean, carrot, strawberry, lettuce, oak, maple, walnut, rose, mint, squash, daisy, and cactus.

In one embodiment, the invention relates to a method for non-destructively sampling individual seeds in a population of seeds comprising: removing contaminate tissue from a seed; screening DNA extracted from the resulting seed after contaminate tissue removal; selecting seeds upon the results of the DNA screening of the resulting seed or a portion thereof; and cultivating plants from the selected seeds. In one embodiment, the contaminant tissue is maternal tissue including but not limited to seed coat or pericarp. In another embodiment, removing contaminate tissue from a seed does not affect the germination viability of the seed. In another embodiment, removing contaminate tissue is performed in such a manner as to keep the remaining tissue of the seed free from pests, and viruses.

In one embodiment, the invention relates to a method of selecting seeds in a population having a desired trait, the method comprising: (a) removing, in a non-destructive manner, a seed chip comprising at least a portion of a coated seed from an individual seed in a population of seeds, (b) separating a coated seed coat from the seed chip; (c) analyzing the seed coat-free seed chip for the presence or absence of at least one trait of interest; and (d) selecting seeds for a seed population based on the presence or absence of at least one trait of interest. The methods can be performed without affecting the germination viability of the seeds if that is desirable. In one embodiment, the coated seed comprises a seed coated with material such as metallic paint, magnetic paint, metal or metallic covering.

In yet another embodiment, the invention relates to a method of selecting seeds in a population having a desired trait, the method comprising: (a) applying a coating to at least a portion of a seed; (b) removing, in a non-destructive manner, a seed chip comprising at least a portion of coated seed coat, (c) separating the coated seed coat from the seed chip; (d) analyzing the coat-free seed chip for the presence or absence of at least one trait of interest; and (e) selecting seeds for a seed population based on the presence or absence of at least one trait of interest. If the germination viability of the seeds is important then the coating is selected so that germination viability of the seed is not affected.

In another embodiment, the method comprises removing at least a portion of contaminate tissue from at least a portion of a seed, such as a seed chip. The seed chip can be obtained using a manual method including but not limited to a scalpel, a knife, or a utility knife. In another embodiment, the seed chip can be obtained using an automatic method including but not limited to a drill, a grinder or a seed chipping device. In some embodiments the material coated on the seed may negatively impact germination viability of the seed. For example, when the seed coat is removed and the seed content is tested in a destruct type of testing parameter, then the coatings impact on viability is not a concern. However, for many methods the material for coating is selected to avoid negatively impacting the seed chip viability of the remaining seed. If a particular material for coating does negatively impact seed viability and seed viability is needed but seed chip viability is not needed, then the coating can be applied only to the section of seed which is seed chip.

In another embodiment, the seed chip and the seed are correlated with each other such that results from analysis of tissue, DNA, protein, oil, starch, and the like from the seed chip can be attributed to the associated seed. For example, a seed chip can be label “SC1” and the seed can be labeled “SE1” thereby allowing the results from analysis of “SC1” to be attributed to a particular seed, “SE1.” The seed chip and the seed can be stored in corresponding wells of microtiter plates (e.g., the seed chip can be stored in A1 of the first plate and the seed can be stored in A1 of the second plate).

Germination viability means that a predominant number of sampled seeds, (i.e, greater than 50% of all sampled seeds) remain viable after sampling. In a particular embodiment, at least about 75% of sampled seeds and in some embodiments at least about 85% of sampled seeds remain viable. It should be noted that lower rates of germination viability may be tolerable under certain circumstances or for certain applications, for example, as genotyping costs decrease, a greater number of seeds could be sampled for the same genotyping cost.

In one embodiment, removing contaminate tissue from a seed comprises applying a coating to the seed and using the coating to separate desired tissue from undesired tissue. This coated contaminate tissue is seed coat or pericarp. The coating can be applied over the entire seed, ¾ of the seed, ½ of the seed, ⅓ of the seed, ¼ of the seed or only a part of the seed that will be used for testing or even a subset of the part of the seed that will be tested. The coating must be applied to allow the contaminate tissue to be attracted and removed from the remainder of the seed material. Depending on how the seeds will be handled further downstream, a partial coating of the seed may be sufficient or an entire coating may be necessary. The coating can be applied before or after any partitioning of the seed occurs. The coating can be applied once or more than once. More than one type of coating may also be used.

The coating can be any substance that can be used to separate desired material from undesired material including but not limited to metallic paint, magnetic coatings, magnetic paints, a metal based coating, a metallic coating, a coating with a positive charge or a coating with a negative charge. Non-limiting examples of metallic paint are Krylon, of magnetic paints are Rust Oleum Metallic primer (Vermon Hills, Ill.), liquid mosaic wall magnetic paint (Scientifics, Tonawanda, N.J.), and magic wall magnetic paint from Kling Magnetics (Chatham, N.J.).

The coating can be a paramagnetic material including but not limited to aluminum, copper, lithium, magnesium, molybdenum, platinum, and tantalum. The coating can be a ferromagnetic material including but not limited to cobalt, iron, nickel, gadolinium, steel. Or any compound that can be used as magnetized metal.

The coating can be applied in any manner that allows the contaminate tissue to be removed from the remainder of the seed material, including but not limited to, spraying, brushing, dipping, electric spraying, soaking, or immersing.

In one embodiment, a coating of metallic paint is applied to the seed. The seed then is placed in a seed chipping device, such as shown in U.S. Pat. No. 7,502,113, or the device in US Application Publication 2010/0050300 which is incorporated in its entirety by reference. The seed chip and the attached and painted seed coat are deposited into a container, such as a well of a microtiter plate. The seed coat is removed from the seed chip quickly manually or automatically by placing an attractant force such as, for example, a magnet, proximate to the coating.

Any type of attractant may be used provided the attractant has sufficient affinity for the coating to allow removal of the coated material. The attractant can take any form including solid, liquid or gas. The attractant and coating may be in immediate contact or in indirect contact. The attractant may be applied once to remove the coated material or more than once. Multiple rounds of application of the attractant may aid in removal of the coated material. Any amount of coated material may be removed as long as the efficiency and accuracy of testing for example genetic analysis is improved including but not limited to from about 1% improvement through to 100% improvement or from about 5% anywhere through to 95% or from about 10% anywhere through to 90% or from about 20% anywhere through to 80% or from about 30% anywhere through to 70% or from about 40% to anywhere through to 60% or from about 50% to about 55%.

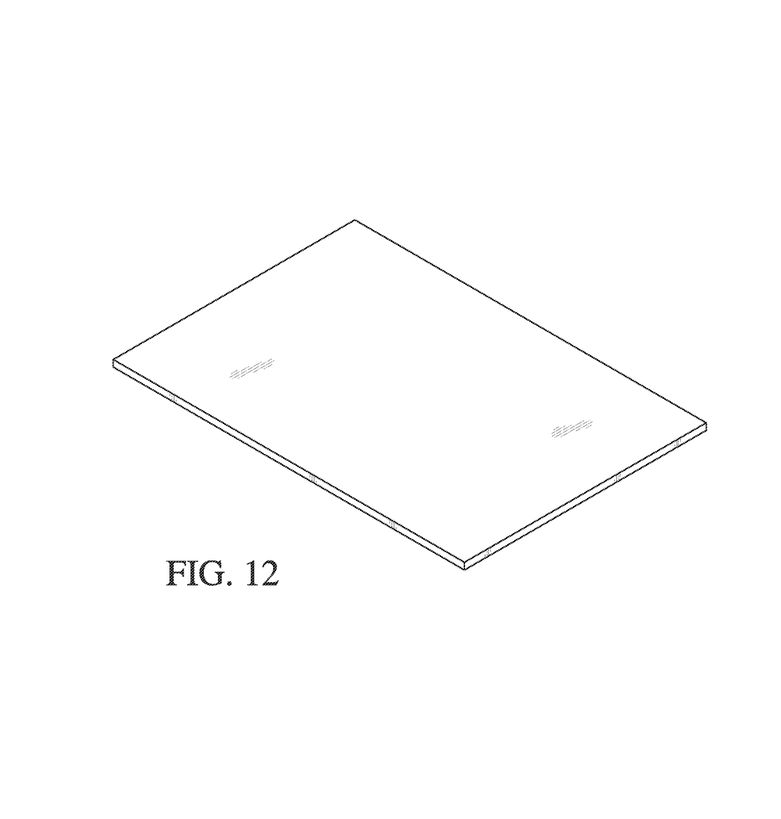

One useful attractant is a piece of metal if the adhered composition is sufficiently strongly magnetized to be extracted with metal. If the seed is coated with magnetic or metallic compositions another useful attractant is a magnet. This can be a block magnet, see FIG. 12. The attractant can be a magnetic force that produced as an electromagnet so the attractant is capable of being switched on and off. Magnets such as a Neodymium (NdFeB) magnet with grades of N48, N50 and N52 are sufficient to attract a lightly coated loosened seed coat. Generally, the magnet strength is related to the “N” number but not always. N52 is the magnet material. A magnet's strength comes from how well it is magnetized, hopefully to saturation. Such neodymium magnets (also known as NdFeB, NIB, or Neo magnet),are rare earth magnets. These are classified as permanent magnets and are made from an alloy of neodymium, iron and boron which form a tetragonal crystalline structure Nd₂Fe₁₄B. This is the strongest, easily found, permanent magnet. But other magnets made of different alloys or electromagnets can also be used. If a magnet is sufficiently strong to attract and extract the coated seed coat from it location near the other seed tissue, then it can be used within the scope of this invention.

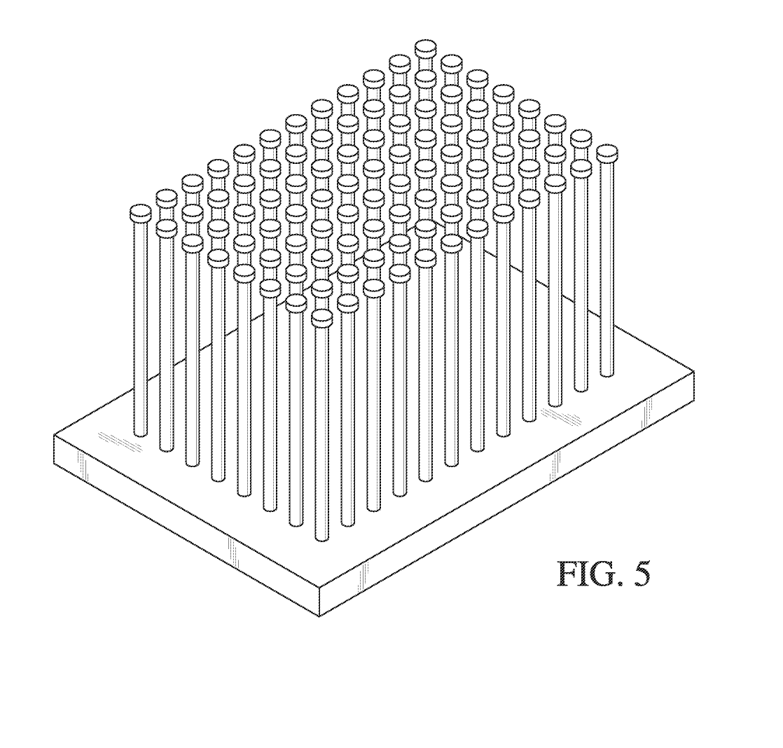

The ability to turn on or turn off the force of the attractant, allows the attracted seed coat material to be more readily relocated. By turning the attractant force off the coated seed coats can be discharged from the magnet into new locations such a within a different well block for testing or in another location that is correlated with the chip/or seed from which the coat was extracted, or in the discard container if the seed coats are not needed. FIG. 5 shows a magnet that is a pronged magnet. This magnet's prongs are adapted to lower into the well area in which the seed chip and coated seed coat are located or stored. The magnetic paint on the seed coat is attracted to the magnet. This attraction allows for extraction of the coated seed coat tissue from the location of the uncoated seed tissue. The FIG. 5 magnet has the same number of prongs as the block has wells. Depending on the storage container for the seed tissue the magnet can be adapted to have different prong number, shape, strength, size and length.

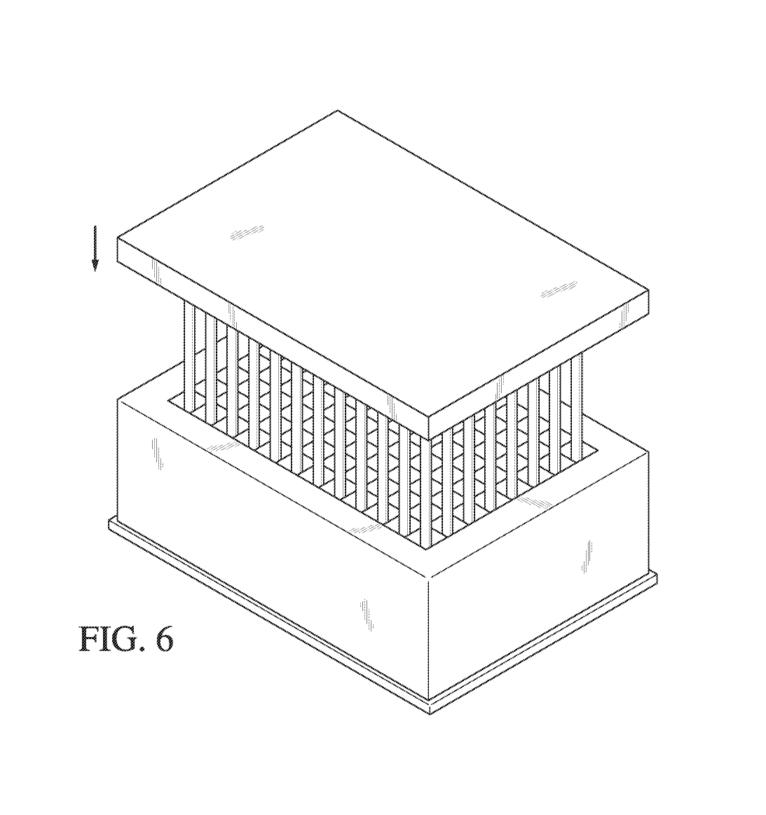

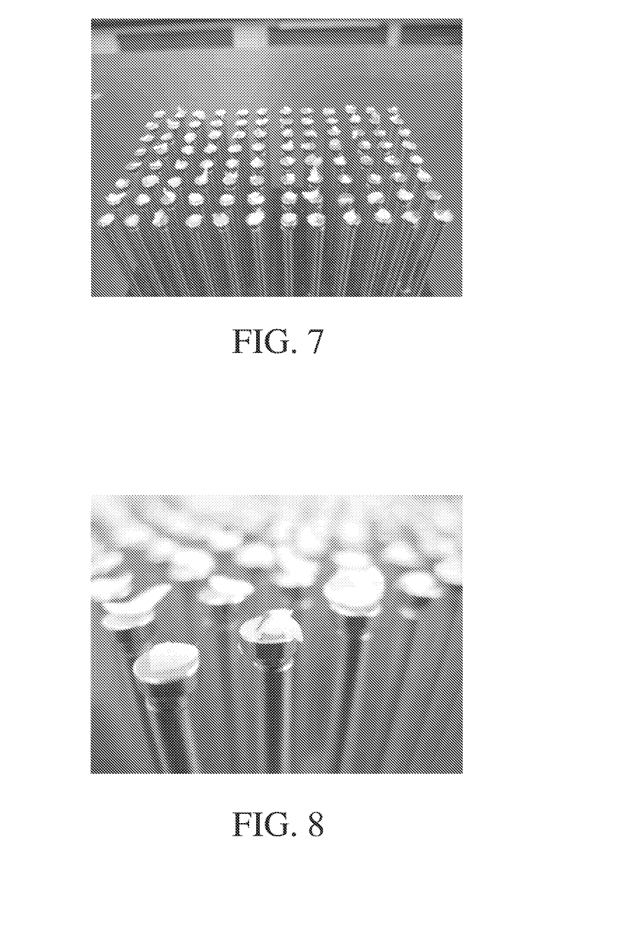

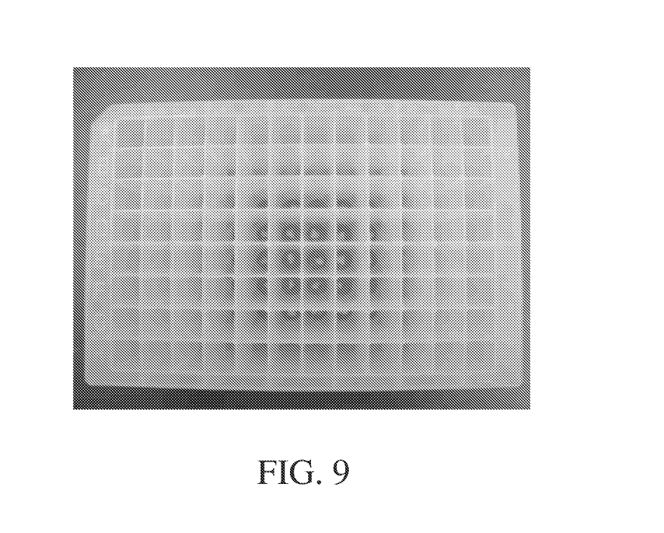

The magnet can have any number of prongs. When automating the removal of the contaminate coated seed tissue from the desired seed tissue it is useful to have magnet prongs that correspond to the number of wells in the block. Alternatively, the magnet can have prongs that correlate with number or width of one row or one columns in the well block. Additionally, the magnet can have sufficient prongs to do more than one block of wells simultaneously or it can be a single prong magnet adapted to extract seed coats on an individualized basis. As shown in FIG. 6, the magnet is lowered into the wells and the detached coated seed coat is attracted to the magnet prongs. As shown in FIGS. 7 and 8 when the magnet prongs leave the wells the seed coat is adhered to the magnet. FIG. 9 shows the wells which now contain only the seed chip without the seed coat contaminate. However, the wells with the uncoated controls will remain in the wells and will not adhere to the attractant and therefore were not extracted by the magnet.

The magnet prongs of FIGS. 7 and 8 each hold an individual contaminate seed tissue in this instance the coated seed coat. The contaminate seed tissue can be relocated into separate microwells in a manner that correlates the seed tissue contaminate with its respective seed chip/seeds. This allows the seed contaminate to be employed for testing for the such information as the seeds maternal genotype.

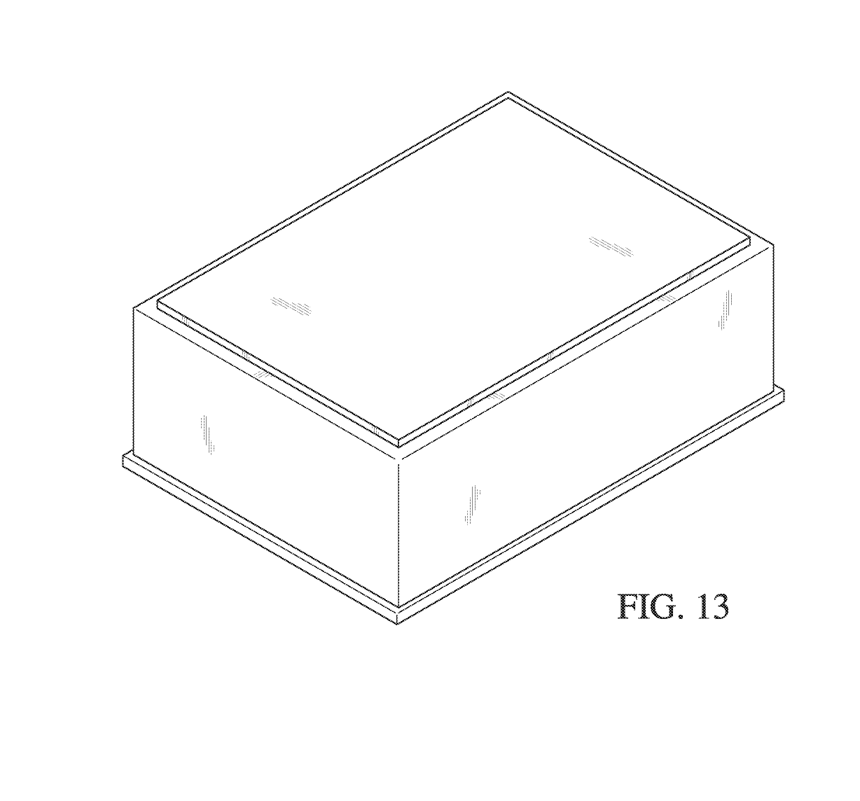

Removal of the coated seed coat can be accomplished in a variety of ways. The principle is to separate the coated portion of the seed, which likely contains the seed coat, from the desired tissue. One simple approach is to cover the entire microtiter plate with a magnet see FIG. 12 and FIG. 13. The microtiter plate (or other collecting device) is inverted upside down several times. This locates the coated seed coats on the magnet in the same pattern as the associated seed chip in the wells of the microtiter plate. To facilitate correlation of the contaminate seed tissue with the desired seed tissue the well pattern can be placed on the magnet making contaminate association with the desired seed tissue more visual.

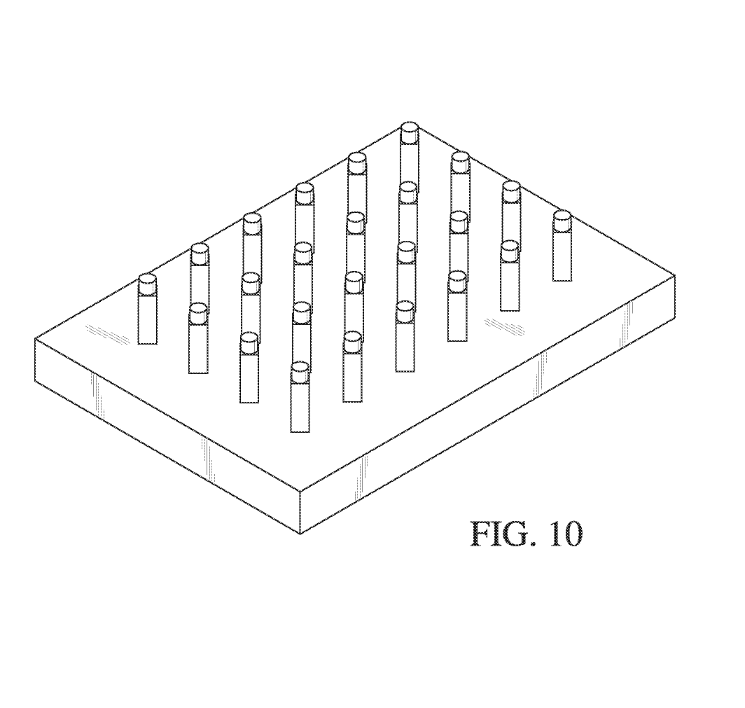

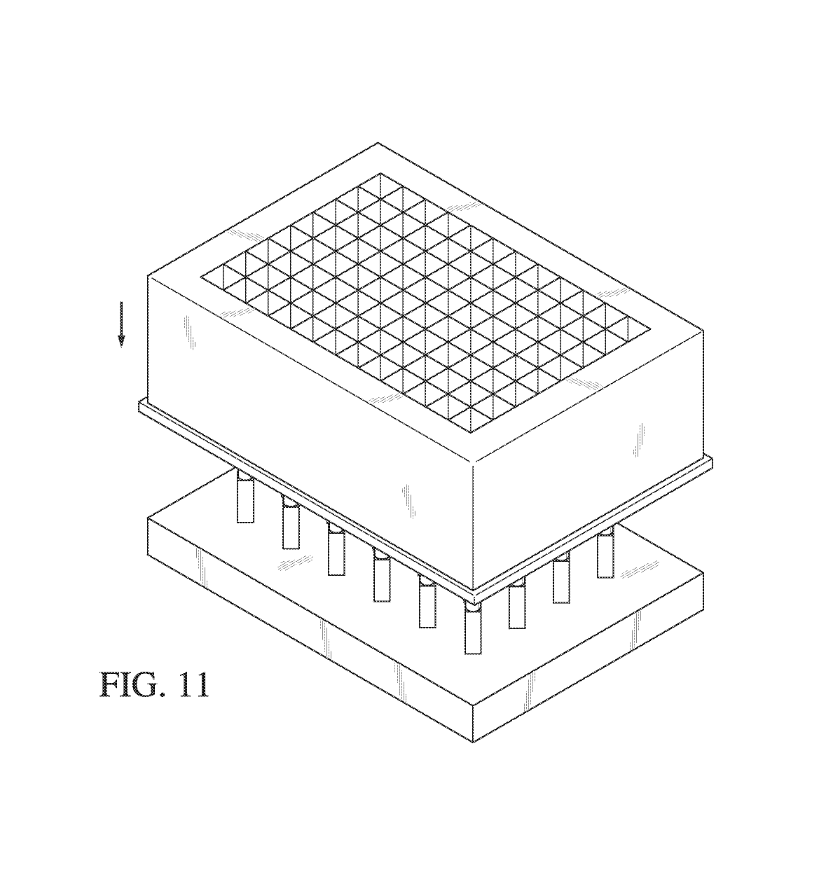

Alternatively, individual magnets can be inserted into a number of the wells, see FIGS. 10 and 11 or each of the wells, see FIG. 5. Alternatively a microtiter plate can be placed upside down on top of the microtiter plate holding the seed tissue and the contaminate seed tissue and the magnet can be applied to the top of the plate drawing the contaminate seed tissue into the second microtiter plate. It would also be possible to transfer the desired tissue into a new holding device by capturing the seed coat with a magnet at the bottom of the original seed chip collecting device, while dumping the desired seed tissue into the new holding device.

These types of seed contaminate handling procedures are particularly suited for high through put automation. This process comprises detaching the seed coats from the seed chips, separating the seed coats from a multitude of seed chips simultaneously then locating these seed chips within a testing apparatus. One method of separating the seed coats from a multitude of seed chips simultaneously employs a robotic arm to locate the attractant proximate the seed tissue holding device. The contaminate seed tissues are withdrawn the arm moves the withdrawn contaminate tissues to a new location and the seed chips are further processed. The seed chips in the holding device, without the contaminate seed tissue, can be processed for testing of the genotype or phenotype characteristics of this seed tissue. Often the testing involves the use of the DNA from seed tissue which is employed for genetic testing. The results are then analyzed and used in various breeding selections.

In another embodiment, the method further comprises loosening contaminate tissue from the seed chip prior to removing the contaminate tissue. Depending on the type of seed, the age of the seed, and method of harvesting the seed, loosening the contaminate tissue may or may not be necessary. One process for loosening contaminate seed tissue is shown in U.S. Pat. No. 7,141,260, which discloses a method of sonicating maize seeds to loosen the pericarp from the remaining seed tissue. A sonication mechanism is used to generate ultrasound energy and impart ultrasound wave energy, which loosen the contaminate seed tissue, such as the pericarp. A frictional mill is then used to mill the sonicated seed to separate the loosened contaminate tissue, such as the seed coat, from the endosperm without damage to the endosperm DNA.

In another embodiment, the coating can be applied before or after the seed is subjected to loosening of the contaminate tissue. If the coating is applied prior to the loosening step then the coating is selected to withstand the loosening process such that the coating remains on the loosened seed coat.

In another embodiment, the coated seed chip, which contains the contaminate tissue, such as the seed coat; can be drawn directly into a new well of a microtiter plate. DNA can be extracted from the contaminate tissue and analyzed to determine the genotype of the maternal plant. Alternatively, the contaminate tissue, such as the seed coat/pericarp, can simply be discarded or it can be tested for other genotypic traits.

In another embodiment, the method comprises extracting DNA from the desired tissue. Any DNA extraction methods known to those of skill in the art, which will provide sufficient DNA yield, DNA quality, and amplification, can be used. A non-limiting example of suitable DNA-extraction methods is SDS-based extraction with centrifugation. In addition, the extracted DNA may be amplified using known amplification methods including but not limited to PCR, and quantitative PCR.

In another embodiment, the method comprises screening DNA for a desired trait or genetic marker. Non-limiting examples of markers include but are not limited to genes, intros, exons, restriction fragment length polymorphisms (RFLP), single nucleotide polymorphisms (SNP), amplified fragment length polymorphisms (AFLP), random amplification of polymorphic DNA (Rapd), real time PCR which determines the presence and/or copy number of genes (including GMOs and ploidy number) and simple sequence repeats (SSR). Additional markers are well known by those skilled in the art and are described in Molecular Cloning: A Laboratory Manual (Third Edition, Cold Spring Harbor Press).

By removing the contaminated tissue, such as seed coat or pericarp, genetic analysis of the remaining seed material is more accurate, resulting in an increase in the number of seeds appropriately selected. In addition, seeds that do not have the genetic marker or trait of interest are easily excluded, and therefore, resources such as time, money, and labor are saved by not planting seeds lacking the desired profile. By practicing the methods disclosed herein, identification of seeds with the desired genetic marker, genotype, phenotype or trait of interest can be increased.

The desired trait may be an entire genetic profile. Alternatively it maybe a phenotype or a genetic locus that is presence as a dominant or recessive allele. In certain embodiments of the invention, the genetic locus confers traits such as, for example, male sterility, waxy starch, pest resistance, herbicide resistance, insect resistance, resistance to bacterial, fungal, nematode or viral disease, yield, lodging resistance, height, maturity, water use efficiency, amylase, resistance to nutrient deficiency, grain composition, and altered fatty acid, phytate or carbohydrate metabolism. The genetic locus may be a naturally occurring gene introduced into the genome of a parent of the variety by backcrossing, a natural or induced mutation, or a transgene introduced through genetic transformation techniques. When introduced through transformation, a genetic locus may comprise one or more transgenes integrated at a single chromosomal location or one or more transgenes integrated at multiple chromosomal locations.

In one embodiment, the detection of the seed's profile, genetic locus or genetic marker is by a method selected from the group comprising allele-specific PCR, gel electrophoresis, capillary electrophoresis, microchannel electrophoresis, polyacrylamide gel electrophoresis, fluorescence detection, fluorescence polarization, DNA sequencing, Sanger dideoxy sequencing, ELISA, mass spectrometry, time of flight mass spectrometry, quadrupole mass spectrometry, magnetic sector mass spectrometry, electric sector mass spectrometry, fluorometry, infrared spectrometry, ultraviolet spectrometry, palentiostatic amperometry, DNA hybridization, DNA microarray, GeneChip arrays, HuSNP arrays, BeadArrays, MassExtend, SNP-IT, TaqMan assay which is useful for allelic determination and real time PCR, Invader assay, MassCleave, southern blot, slot blot, and dot blot and the like.

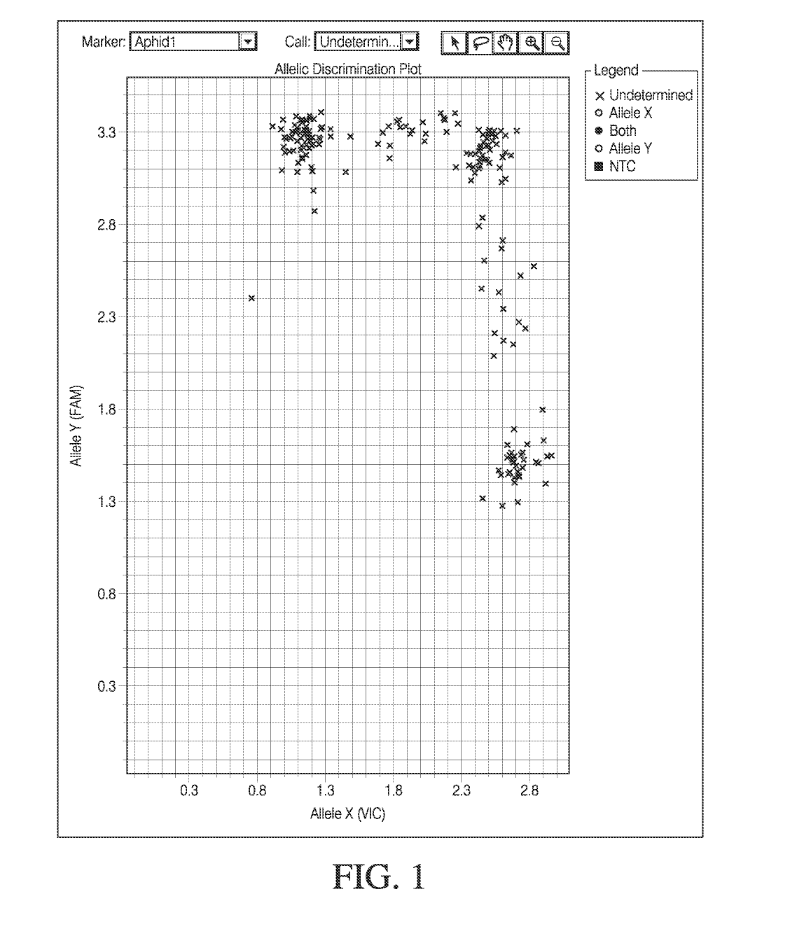

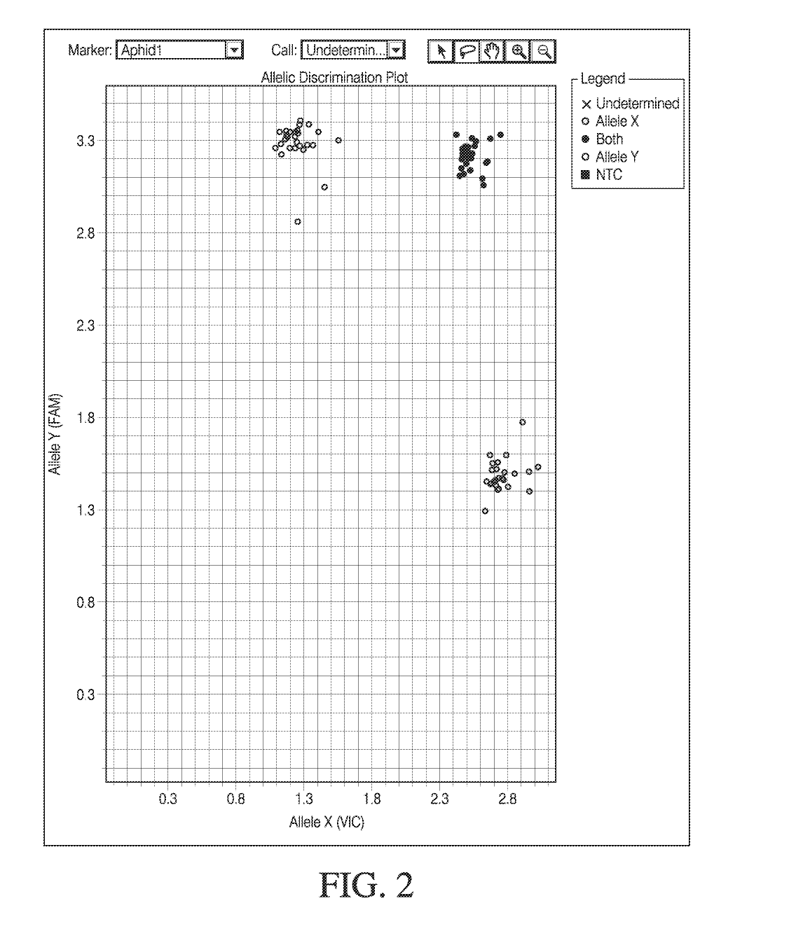

Turning to FIG. 1 this figure shows an allelic discrimination plot from soybean seed chips with seed tissue contaminate. DNA was extracted from seed chips with seed coat material. Homozygous and heterozygous alleles are not clearly distinguishable. Due to the breadth of the heterozygous pattern, it is unclear which seed lines are actually homozygous. Based on this plot, one of ordinary skill in the art would not be able to identify the points on the graph that correspond to all of the truly homozygous test results and all of the heterozygous test results. The maternal DNA of the contaminate seed tissue skewed the results of this plot; and an automated analysis was not possible. The FIG. 1 plot can be compared with the FIG. 2 plot. The FIG. 2 plot was the sample allelic discrimination plot as shown in FIG. 1 however, this plot was run on seed chip DNA from which the contaminate seed tissue, the soybean seed coat, was removed before DNA extraction. This plot taken with seed chip that lack the seed coat shows a clear division of what seed were homozygous material and what seeds were heterozygous.

These plots in FIGS. 1 and 2, with maternal DNA and without maternal DNA respectively, show the skewing effect that the seed coat produced. The methods of the invention eliminate this DNA contaminate in a high-throughput, automated fashion by coating the seed with a coating and loosening the seed coat, when necessary, by freeze drying, sonication or heat. Once the seed coat is loosened the coated maternal tissue shown in FIG. 2 was removed by application of an attractant. In this case the coating was magnetic paint and the attractant was a magnet.

The coatless seed chip and the removed seed coat both have DNA that can be tested and used. Genetic material in the seed chip is useful for screening for plant ploidy, recurrent parent alleles in a backcross breeding program, for homozygousity in a bulk breeding population, for selection or detection of transgenes, native alleles or parental alleles. The identification of seed genotypes or phenotypes before planting allows for the identification and selection of seeds carrying the desired traits, and therefore, allows unwanted seeds to be culled.

By using the methods of the invention to remove maternal tissue from the seed chip prior to the PCR testing, the test results are more accurate and selection of seed is improved. Removing the seed coat, and hence seed coat DNA, makes analysis of the endosperm easier. The method allows accurate seed endosperm zygosity to be determined without the skewing effect of the seed coat. The ability to accurately detect the desirable seeds results in a reduced the number of rows of seeds per population being planted in a breeding program. Since less seeds are misidentified, fewer desired seeds are planted, more time and field space is efficiently used and the breeding or conversion program has field space to increase the number of populations tested. This improvement in the accuracy of the testing, results in improved land usage reduced land requirements and decreased labor costs, etc.

The methods of the present invention may be further applied to identify hybrid seed for transgene testing. For example, in a conversion of an inbred line at the BCₙF1 stage, a hybrid seed lot was 50% hemizygous for the trait of interest and 50% homozygous for the lack of the trait. In order to generate hybrid seed for testing, this material without its pericarp contaminate seed tissue should be screened to identify the F1 seeds that are hemizygous. Such a seed contaminate tissue separation method is advantageous in that yield data from only the hybrids with genetics of the correct trait zygosity, could be selected and analyzed. This data would be available prior to having seeds that could produce a field of plants capable of producing this zygosity.

The invention provides an apparatus and method that allows for seed chip testing and identification of the appropriate seed blocks having or not having the specific desired trait, marker or genotype. The seed chip, particularly a corn, melon, pumpkin or soybean seed chip, will allow for the removal of seed coat tissue from seed chips in a high throughput fashion.

In operation of the method of the present invention seeds are lightly coated. This coating can cover the entire seed surface with the attractant or just a portion of the seed surface. One of the coatings that is readily available is magnetic paint. Any manner of applying a thin layer of paint to a portion of the seed's outer surface can be employed. For example, the seed can be rolled in paint, brushed, dabbed or sprayed with the coating material. In one experiment, Krylon magnetic paint was sprayed on the seed outer surface so thinly that the soybean's hilum remained visible. This thin coating of magnetic paint was sufficient coating to allow for seed tissue separation to occur.

The seed's painted surface is allowed to dry before a small chip of the seed material was removed leaving a viable remnant of seed. The seed chip contains seed material and a lightly painted seed coat attached to the chip's outer seed surface. Equipment for chipping small pieces of seed from larger pieces is known. Seed has for years been chipped manually by cutting, sawing, drilling, slicing snipping, the seed with everything from nail clippers, to paper cutters, to scissors, knives, drills, wire cutters, saws and the like. Any instrument that could remove a piece of seed could be employed. These manual seed chipping methods have become automated, because seed testing has become more rapid and efficient. For example, an automated laser chipping device such as shown in WO 2010/022286, and, an automated brocaded saw device shown in EP1991043 and its US counterparts have automated previously manual seed chipping procedures. Automated chipping through use of a high speed saw, electric knife, drill, or laser often produce high temperatures in the chip, which results in the seed and the seed coat tissues become tightly molded to one another. Because seed coat tissue can skew results of seed testing, detachment and removal of the seed coat prior to seed chip testing is desirable. High temperature molding, produced by automated chipping makes loosening or detachment of the seed coat from the seed chip problematic,

The known methods of loosing seed coats include exposing seeds to elevated oven temperatures, prolonged soaking in water, and sonification of the seeds. Another known method of detaching the seed coat from the seed chip tissue is by placing the chip (or if the seed itself is not being preserved for germination, the seed) into liquid nitrogen. Use of liquid nitrogen for detaching the seed and the coat material is expensive and requires special chemical storage and treatment. The method for using nitrogen is shown in W. John Mullin et al., J. Agric. Food Chem., 2001, 49 (11), pp 5331-5335.

The present invention comprises a newly discovered protocol to promote loosening of the contaminate seed tissue from the seed tissue. The application of a few μl of 100% ethanol to the attached contaminate seed tissue and the seed tissue loosened the two tissues. Only a small amount of ethanol, sufficient to cover the chips when in a microwell quickly interacts with the molded seed chip and coat to detach and loosen the coat and cotyledon tissue. It was surprising to discover that by adding a volatile liquid like alcohol would induce the seed coat to detach from the seed tissue. The volatile liquid can be diluted with a liquid of low vapor pressure (e.g. 70% ethanol). And a number of different types of alcohols can be used if these chemicals do not negatively affecting the ability of the desired seed tissue to be processed and/or tested. Volatile liquids such as acetone, isopropyl alcohol, propanol, methanol, butanol and the like could be employed without undue experimentation.

In the present inventions method of contaminate seed tissue loosening from the seed tissue, the ethanol was evaporated from the microwells holding the seed tissues at room temperature under a hood. A more efficient process may employ an oven at elevated temperature to facilitate a more efficient removal of the ethanol from the microwells. The ethanol efficiently loosens the seed coat from the seed chip leaving two separate seed tissue portions in the microwell: the maternal DNA present in the seed coat portion, and the seed chip portion made of the cotyledon tissue.

Another protocol employs water to detach the seed coat and cotyledon tissue. This is not a preferred process because both seed tissues tend to swell making them larger and heavier. The water weight in the contaminate tissue require the coating to be thicker, using more magnetic paint and a stronger magnet to lift the weight of the contaminate seed tissue. However, one partial solution to the the water weight concern, is to evaporated the water from the well and the seed coat.

The present invention in operation, captures the magnetized contaminate seed tissue with a magnet. This allows the contaminate seed tissue to be location in a different location then the desired seed tissue. Initially, seeds were coated heavily with magnetic paint, but it was discovered that lightly coated seeds were very useful and lighter.

Soybean seeds coated with magnetic paint were chipped, and the chips were transferred into a 96 well block. The block could then placed into an oven at 65 C to separate seed coat tissue from cotyledon tissue. The seed chips that were magnetized with magnetic paint had their seed coat loosened in the oven heating process, then the loosened seed coats were subjected to an attractant force, in the form of a magnet. Alternatively, the loosening could have been through the ethanol protocol of the present invention, regardless of the process of loosening the two seed tissues, the coating on the one tissue allows for the seed tissue separation.

The seed tissue separation shown in these figures involve a magnet with individual prongs adapted to slip within the open portion of the microwells. The magnetic or metallic paint on the seed coat tissue, is attracted to the magnet, and of course, the inner seed chip tissue which lacks the paint is not attracted to the magnet. Thus the detached seed coats which magnetically engage with the magnet or metal can then be lifted out of the 96 well block leaving the cotyledon seed tissue in the wells. FIG. 6 shows the magnetically engaged seed coats after separation from the detached seed tissue. The coated seed coats were attached to the magnet; see FIG. 8 in the same pattern as the associated seed chip in the wells. This allows the separated seed tissue material to be correlated with each other or with the remnant seed from which the material was taken.

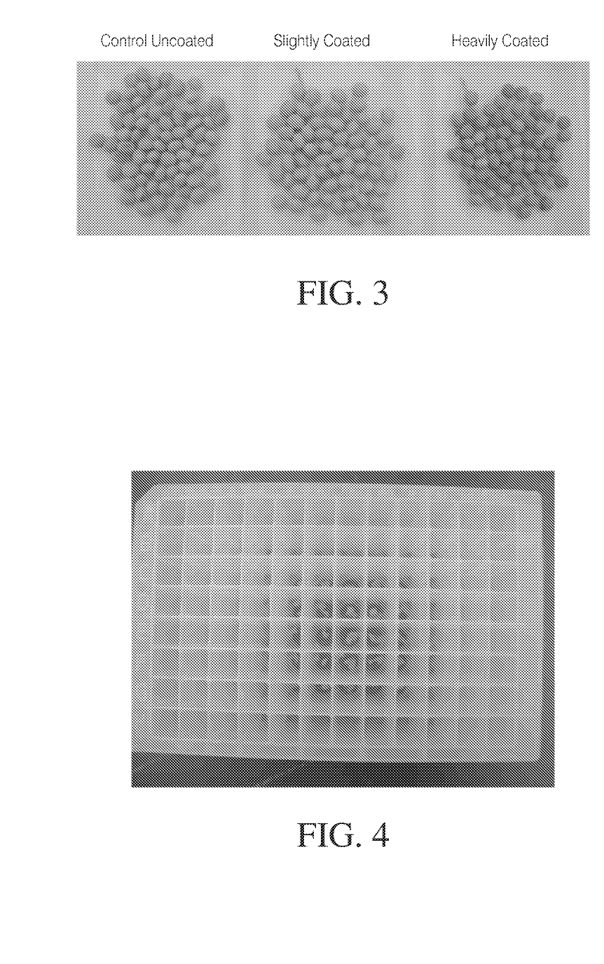

In general, FIGS. 3-9 show steps of separation of soybean contaminate tissue and cotyledon seed tissue with the magnetized system of the present invention. These contaminate tissues could be released into a second correlated vessel, if the magnetic force of the magnet is released while the magnet with the seed coats is positioned proximate this vessel. The vessel could be a second microwell titer plate.

The Figures show an alternative plate like magnet that could be employed in this system instead of the pronged magnet. The pericarp could be separated with a large magnet that covers and the entire plate, acting as a lid to the well plate. The plate of wells could be inverted to place the coated pericarps in close proximity to the magnet plant. The microwell titer plate with large lid like magnet can then be righted. The coated pericarp material would be adhered to the magnet and the seed chips would be in the wells.

These Examples are provided for the purpose of illustration only and the invention should in no way be construed as being limited to these examples, but rather should be construed to encompass any and all variations that become evident as a result of the teaching provided herein.

### EXAMPLES

### Example 1

### Treatment of Soybeans

Soybean seeds are lightly coated over the entire seed surface with magnetic paint. A very thin coating of Krylon magnetic paint spray is applied from a distance of approximately 15 inches between seed and spray nozzle.

After the paint dried, a small chip is removed from each of the seeds. The selection of a seed chip or sample could be achieved by placing the seed within an automated seed cutting device such as shown in U.S. Pat. No. 7,502,113, which is incorporated in its entirety by reference. The seed chips are collected in a microwell titer plate.
1. The coated seed chips may have seed coat and seed tissue adhered to each other but in this instance a razor was employed to separate the seed chip from the seed. These materials are separated by placing the wells/blocks (or other collecting devices that are employed) into an oven at 65-75° C. Dehulling of soybean seeds via these high temperatures is a standard procedure used by the food processing industry. The coated seed chips can also be dried at a temperature somewhat higher or lower than 65-75° C. if it detaches the hull or coat.
2. In other experiments which uses a laser to cut the seed, the seeds are placed in an oven, or a freeze dryer/food processor to detach the coated seed coat from the remaining seed tissue. The use of the high temperatures or a freeze dryer will work on approximately 70% of all samples to dissect the seed coat from the seed chip.
3. If the cotyledon and coat tissue are still attached to each other, the elevated temperature and the freeze dryer process can be combined. Additionally, either or both of these processes of detaching the coat can be combined with or followed with a shaking step. The microwell titer plate can be placed in a shaker machine for additional detachment of the coat from the chip. For a more efficient dissection of the two tissues all three of these steps can be employed and or combined to detach the seed and the seed coat.
4. A slightly higher level of dissection of the two tissues is possible if after the use of the freeze dryer or the oven the microwells are shaken vertically. However, use of this dissection process may result in a few escapes.
   Step 2: Capturing the Magnetized Seed Coat with a Magnet and Removing them from the Seed Chip location

The seeds coats, which are magnetized with magnetic paint and loosened, are removed out of the wells in the block by application of an attractant. In this experiment, the magnetic paint on the seed coat is attracted by a magnet. Coated seed coat removal can be performed in various ways: One simple approach is to cover the entire block with a magnet. The block is inverted (or other collecting device) upside down a couple of times. This locates the coated seed coats on the magnet in the same pattern as the associated seed chip in the microwells.

In this experiment, individual magnets shown in FIG. 6 are inserted into each well. FIG. 6 shows the multitude of magnet prongs which could be used in this experiment. FIGS. 6-13 show different forms of magnets that make up different embodiments of the present invention. Each of these types of magnets can be used as is shown in the figures.

This magnet is well adapted for use in an automated system. The magnet can be robotically lowered into the wells and the detached seed coat is attracted to the magnet prongs.

In this experiment the prongs were manually lowered into the wells to within a fraction of reaching the bottom of the wells. When the magnet prongs are drawn out of the individual wells the seed coats adhered to the magnet, and the seed chips remained within the wells.

The seed coats were relocated into a separate location from the chip or seed location. The seed coats could be positioned into another microwell plate which has wells correlated to the seed chip/seeds from which the coats are detached. These seed coats could then be used for further testing. Or as in this instance, the coats can be relocated by being wiped from the magnet prongs' tips and discard.

Alternatively, a 96 well plate can be placed on top of the seed coat/seed chip holding plate and the magnet can be applied to the bottom of the top wells drawing the coated seed coats up into the second well plate away from the seed chips. It would also be possible to transfer the cotyledon tissue into a new holding device by capturing the seed coat with a magnet at the bottom of the original seed chip collecting device.

The microtiter plate, with the seed chips and without the confounding seed coats, is then placed within an automated PCR system for genotypic testing. The results of the genetic analysis will be scored and seeds that correlate with the seed chips having the desired genotypic results are to be selected for further cultivation and harvest.

### Experiment 2

The steps of experiment one could be employed. The seed coats from this experiment and could be employed to determine if the light coating of magnetic paint eliminates the ability to use the seed coat for testing purposes. One test would be to see if DNA extraction was complicated after it is coated with the attractant. The separated coats could be placed in an automated PCR system to determine if the maternal DNA could be assayed for information on the maternal parent or traits from the maternal parent. The seed coat DNA when it is tested should provide the expected maternal parent DNA genotype even with the light coating of magnetic paint.

### Experiment 3

The seeds are in three sets: one is an unpainted seed control, one seed set is lightly sprayed with magnetic paint, and the other seed set is lightly sprayed with metallic paint. The seed chips were cut from the seed with a razor blade. A second set of three chips are chipped with a laser and the seeds are planted in the green house to determine the effect of the paint on the viability of the seed to produce viable plants. The plants were checked to determine if there is normal seedling produced by the cut and coated seeds. The emergence and growth of the seedlings was not significantly different then the emergence and growth of the set of unpainted seed controls.

Although specific embodiments have been illustrated and described herein, it will be appreciated by those of ordinary skill in the art that any arrangement that is calculated to achieve the same purpose may be substituted for the specific embodiments shown. This application is intended to cover any adaptations or variations that operate according to the principles of the invention as described. Therefore, it is intended that this invention be limited only by the claims and the equivalents thereof

What is claimed is:

1. A method of selecting seeds in a population having a desired trait, the method comprising:
(a) applying a coating to at least a portion of a seed in a population of seeds;
(b) removing, in a non-destructive manner, a seed chip comprising at least a portion of seed coat from the coated seed;
(c) separating the seed coat from the seed chip;
(d) analyzing the coat-free seed chip for the presence or absence of at least one trait of interest; and
(e) selecting seeds based on the presence or absence of at least one trait of interest.

2. The method of claim 1, wherein removing, in a non-destructive manner, a seed chip comprising at least a portion of seed coat from an individual seed does not affect germination viability.

3. The method of claim 1, wherein the seed is a soybean or maize seed.

4. The method of claim 1, wherein the coating is responsive to magnetic forces.

5. The method of claim 1, wherein the coating is a magnetic paint.

6. The method of claim 1, wherein separating the seed coat from the seed chip comprises using an attractant that binds to the coating.

7. The method of claim 1, wherein analyzing the coat-free seed chip comprises extracting DNA from said coat-free seed chip.

8. The method of claim 7, wherein extracting DNA from said coat-free seed chip produces DNA that has substantially less maternal DNA than does the seed chip.

9. The method of claim 1, wherein separating the seed coat from the seed chip comprises positioning a number of seed chips within a testing apparatus and separating seed coats from a multitude of seed chips simultaneously.

10. The method of claim 1 comprising loosening the seed coat from the seed prior to removing the seed chip.

11. The method of claim 1 comprising loosening the seed coat from the seed chip prior to separating the seed coat.

12. The method of claim 11, wherein loosening the seed coat comprises sonicating the seed chip.

13. The method of claim 12, further comprising applying heat at levels that do not substantially alter the trait being tested.

14. The method of claim 1 further comprising cultivating plants from the selected seeds and optionally comprising harvesting seed from the cultivated plants.

15. A method for separating a seed contaminate tissue from at least a portion of seed tissue comprising: (a) applying a coating to at least a portion of a seed; (b) loosening seed contaminate tissue from at least the portion of the seed tissue; (c) separating the coated seed contaminate tissue from at least a portion of said seed tissue; and, (d) retaining for further use the remainder of the seed after removing the coated seed contaminate tissue.

16. The method of claim 15, wherein loosening said comprises a step of seed contaminate and seed tissue immersion in a liquid.

17. The method of claim 16, wherein removing the coated seed contaminate tissue comprises using an attractant.

18. The method of claim 18, wherein the attractant is a magnet.

19. The method of claim 15, wherein loosening the seed contaminate tissue from the seed tissue employs at least one step of: sonification, high temperature exposure, alcohol contact, nitrogen contact or shaking.

20. A method of analyzing seed material for a genotype or phenotype characteristic detached from its seed coat, the method comprising: (a) coating seed, (b) loosening, in a non-destructive manner, at least a portion of coated seed coat from seed material, (b) separating, by use of attractant force, the coated seed coat or portion thereof from the seed material; (c) analyzing the coat-free seed material for the presence or absence of at least one genotype or phenotype characteristic.
